(19) United States
(12) Patent Application Publication
(10) Pub. No.: US 2006/0241182 A1
(43) Pub. Date: Oct. 26, 2006

(54) Antibiotic, functional cosmetic and functional food containing levulinic acid and their derivatives

(76) Inventors: Hong-Ki Jun, Pusan (KR); Ung-Chan Yoon, Pusan (KR)

Correspondence Address: LUCAS & MERCANTI, LLP, 475 PARK AVENUE SOUTH, 15TH FLOOR, NEW YORK, NY, 10016, US

(21) Appl. No.: 11/455,417

(22) Filed: Jun. 19, 2006

Related U.S. Application Data
(62) Division of application No. 10/314,508, filed on Dec. 9, 2002.

(30) Foreign Application Priority Data
Dec. 10, 2001 (KR) 2001-77954
Oct. 25, 2002 (KR) 2002-65501

(51) Int. Cl. A61K 31/225

(52) U.S. Cl. 514/547

(57) ABSTRACT

The present invention relates to antibiotics, functional cosmetics and functional foods comprising levulinic acid or its derivatives as effective ingredients. More particularly, the present invention relates to antibiotics comprising levulinic acid or its derivatives having antibiotic activities against various microorganisms including Gram positive bacteria, Gram negative bacteria, yeast and drug-resistant bacteria. Also, the present invention relates to functional cosmetics and functional foods comprising the same.

### CROSS-REFERENCE TO RELATED APPLICATIONS

This patent application is a divisional of U.S. Ser. No. 10/314,508 filed Dec. 9, 2002, which claims the benefit of priority from Korean Patent Application No. 2001/77954 filed Dec. 10, 2001 and Korean Patent Application No. 2002/65501 filed Oct. 25, 2002, the contents of each of which is incorporated by reference herein in its entirety.

### FIELD OF THE INVENTION

The present invention relates to antibiotics, functional cosmetics and functional foods comprising levulinic acid and its derivatives as effective ingredients. More particularly, the present invention relates to broad-spectrum antibiotics comprising levulinic acid or its derivatives having antibiotic activities against various microorganisms including Gram positive bacteria, Gram negative bacteria, yeast and drug-resistant bacteria. Also, the present invention relates to functional cosmetics and foods comprising the same.

### BACKGROUND OF THE INVENTION

Many antibiotics have been developed in order to treat diseases caused by various microorganisms. Various kinds of antibiotics have been developed besides the first antibiotic, penicillin, and are still under development.

After these antibiotics have been used to treat diseases caused by microorganisms, new bacteria having resistance against such antibiotics come out and thus, it is necessary to be developed new antibiotics. However, nowadays, the rate of the advent of new drug-resistant bacteria exceeds the rate of the development of new antibiotics. Among various antibiotics developed previously, vancomycin was considered to be the last antibiotic that can treat methicilin-resistant Staphylococcus aureus and infections caused by other Gram positive bacteria. However, recently, the advent of new bacteria having resistance against vancomycin is being reported in USA, Japan and Europe.

Therefore, in order to overcome the potential serious problems regarding such drug-resistant bacteria in advance, the development of new antibiotics is highly required.

Recently, along with the elucidation of antibiotics resistance mechanisms, the development of antibiotics for new targets involved in the bacterial pathogenesis processes including adhesion and invasion is expected to open a new field in treating infectious diseases. However, recently, compared with the rate of advent of microorganisms having resistance against antibiotics, the development of new antibiotics is not sufficient. In fact, among the antibiotics which have recently been approved or are under examination, only a few are unique.

Hereupon, through the researches for screening new type of antibiotics, the present inventors discovered that levulinic acid and its derivatives show antibiotic activities. Levulinic acid and its derivatives are new antibiotics which are completely different in structure from the known antibiotics and show antibiotic activities to broad-spectrum microoganisms including Gram positive and Gram negative bacteria, yeast and bacteria having resistant against other known-antibiotics.

Levulinic acid is a compound also called β-acetylpropionic acid or 4-oxopentanoic acid which is colorless plate or leaflets with mp. 33-35° C. It is obtained by boiling starch or sugar with hydrochloric acid, and is commercially produced from low grade cellulose by heating with organic acid. Levulinic acid can be produced cost effectively and in high yield from renewable in a new industrial process.

Levulinic acid and its derivatives has found use in highly diverse areas such as chiral reagents, biological active materials, polyhydroxyalkanoates, polymers, antifouling compounds, personal care products, lubricants, adsorbents, printing, inks, coatings, photographs, batteries, drug delivery and corrosion inhibitors. Furthermore, it is also used as a starting material of methyltetrahydrofuran, β-aminolevulinic acid, diphenolic acid [J. J. Bozell et al., Resources, Conservation and Recycling, 28, 227-239, 2000]. However, its use as an antibiotic has not been made public yet in spite of it wide applications.

Leading to the present invention, the inventors found out that levulinic acid and its derivative show antibiotic activities against Gram positive bacteria, Gram negative bacteria, yeast and drug-resistant bacteria and thus, can be used as new types of antibiotics, and as a ingradients for functional cosmetics and functional food to complete the present invention.

### SUMMARY OF THE INVENTION

An object of the present invention is to provide uses of levulinic acid and its derivatives.

The present invention provides antibiotics comprising levulinic acid or its derivatives as effective ingredients.

Also, the present invention provides functional cosmetics comprising levulinic acid or its derivatives as effective ingredients.

Furthermore, the present invention provides functional foods comprising levulinic acid or its derivatives as effective ingredients.

### DETAILED DESCRIPTION OF THE PREFERRED EMBODIMENTS

The present invention is described in detail as set forth hereunder.

I. Antibiotics.

The present invention provides antibiotics comprising levulinic acid or its derivatives, represented by following formula 1.

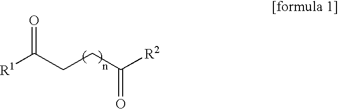

(wherein, R¹ is C₁-C₄ straight or branched alkyl group, or C₃-C₇ cycloalkyl group; R² is —OH, —OR³, —NH₂ or —NR⁴; R³ is C₁-C₄ straight or branched alkyl group; R⁴ is C₁-C₄ straight or branched alkyl group or C₅-C₆ methylene group (—(CH₂)₅₋₆)—) and n is an integral of 1 to 3. Preferably, R¹ is methyl group, R² is —OH and n is 1.)

Levulinic acid is commercially available and its derivatives represented by above formula 1 are prepared by known methods in various and useful ways.

Levulinic acid or its derivatives have strong antibiotic activities against drug-resistant bacteria and other bacteria. Particularly, levulinic acid or their derivatives have excellent antibiotic activities against common-antibiotic-resistant bacteria such as methicillin-resistant Staphylococcus aureus (herein after referred to as MRSA), drug-resistant Pseudomonas aeruginosa and vancomycin-resistant Enterococcus (VRE). Levulinic acid or its derivatives have excellent antibiotic activities against bacteria such as Staphylococcus aureus and Enterococcus faecium. Additionally, levulinic acid or its derivatives have antibiotic activities against yeast.

Levulinic acid or its derivatives have effective antibiotic activities against bacteria infecting the finny tribe, for example Lactococcus garvieae, Edwardsiella tarda and Vibrio anguillarum. Levulinic acid or their derivatives have excellent antibiotic activities against tooth-decaying bacteria such as Streptococcus mutans, Streptococcus sobrinus-coykendal, Streptococcus doweni and Streptococcus salivarius. Levulinic acid or their derivatives have excellent antibiotic activities against Salmonella typhimurium, pathogen in poultry and Propionibacterium acnes KCTC 3326, pimple-causing bacteria. As stated above, levulinic acid or their derivatives have antibiotic activities against various microoganisms. (Table 1-5)

Levulinic acid or their derivatives are used as antibiotics against common-antibiotic-resistant bacteria such as methicillin-resistant Staphylococcus aureus, drug-resistant Pseudomonas aeruginosa and vancomycin-resistant Enterococcus, or used as therapeutics and preventor against diseases caused by bacteria involved tooth decay, generation of pimples and pathogenesis for animals including domestic animal, poultry and finny tribe.

Levulinic acid or their derivatives of the present invention can be administered as various formulations such as oral and perenteral formulations. The above formulations are prepared by using common vehicles or diluting agents such as packing agents, bulking agents, bonding agents, wetting agents, disintegrating agents and surfactants. Solid formulations for oral administration are tablets, pills, powders, granules, capsules and troches. These solid formulations are prepared by mixing more than one of the compounds represented by formula 1, with simple vehicles for example, starch, calcium carbonate, sucrose, dextrose, lactose and gelatin. In addition, lubricants such as magnesium stearate, talc are used with the mentioned simple vehicles.

Liquid formulations for oral administration are suspensions, liquids, emulsions or syrups. These liquid formulations are prepared by mixing one or more of the compounds represented by formula 1, with various vehicles for example, wetting agents, sweetening agents, aromatic agents and preservatives, in addition to water or liquid paraffin.

Formulations for perenteral administration are prepared by mixing one or more of the compounds represented by the above formula 1, with additives such as sterilized water, non-aqueous solvents, suspensions, emulsions or auxilaries. Non-aqueous solvents and suspensions are selected from a group comprising propylene glycol, polyethylene glycol, vegetable oil as olive oils and injectable esters as ethyl oleate. Bases of auxilaries are selected from a group comprising witepsol, macrogol, tween 61, cacao butter, laurin butter, glycerol and gelatin.

Administration dosage of the compound, represented by above formula 1 is dependant on patient's condition, for example age, weight, sex, hygienic condition and seriousness of disease. If drug is administered to adult patient weighing 70 Kg, administration dosage is generally 1 mg˜10 g per 1 day, preferably 1 mg˜5 g per 1 day. According to diagnosis of doctor or pharmaceutist, drug can be administrated to patient once or many times at regular intervals.

II. Functional cosmetics

The present invention provides antibiotics comprising levulinic acid or their derivatives, represented by above formula 1.

Levulinic acid or their derivatives, represented by the above formula 1 have antibiotic activities against bacteria or yeast. Therefore, levulinic acid or their derivatives are used for preparing functional cosmetics having inhibitory activity against various bacteria causing pimple and skin trouble. For example, levulinic acid has antibiotic activity against pimple-causing bacteria such as Propionibacterium acnes KCTC 3326, or pimple pyogenic bacteria such as Staphylococcus aureus, Pseudomonas aeruginosa. Therefore, functional cosmetics having efficiency in preventing or treating pimple are prepared by using levulinic acid or their derivatives.

The present invention provides functional cosmetics such as face lotions and creams, comprising levulinic acid or their derivatives, represented by above formula 1.

Face lotions of the present invention are prepared by adding antibiotic materials such as levulinic acid or their derivatives, to common composition ingredient such as abrader, purified water, glycerin, buffering agents, aromatics, oils, alcohols and etc.

According to the present invention, content of the effective component such as levulinic acid or their derivatives in fresh lotions, depends on the absorptivity, availability and excretion rate of the active component at the skin as well as age, sex and condition of user. Preferably, the effective component contains 0.01˜5.0% of the total weight of fresh lotion. If the effective component is contained below 0.01% of total weight, antibiotic activity of fresh lotion is reduced. If effective component is contained above 5.0% of total weight, antibiotic activity of fresh lotion is not increased despite-increase of content.

Creams of the present invention are prepared by adding antibiotic material, levulinic acid or their derivatives to common composition ingredient such as abrading agent, purified water, glycerin, buffering agent, aromatics, oils, alcohols and etc. According to the present invention, contents of effective component in fresh lotions, are dependent on absorptivity, activity and excretion rate of active component in skin as well as age, sex and state of user. Preferably, effective component is contained 0.01˜5.0% of total weight of fresh lotion. If effective component is contained below 0.01% of total weight, antibiotic activity of fresh lotion is reduced. If effective component is contained above 5.0% of total weight, antibiotic activity of fresh lotion is not increased despite increase of content.

According to the present invention, functional cosmetics comprising levulinic acid or its derivatives, have non-toxic effect to normal skin with the application of cosmetics for 20 days and thus, the above mentioned cosmetics are innoxious within general applications.

III. Functional Foods

The present invention provides functional foods comprising of levulinic acid or its derivatives as effective ingredients.

Levulinic acid or its derivatives, represented by the above formula 1 have antibiotic activities against bacteria or yeast. Therefore, levulinic acid or their derivatives are used for preparing functional foods preventing diseases caused by bacteria or yeast. For example, levulinic acid and its derivation have antibiotic activity against Helicobacter pylori causing stomach ulcer, duodenal ulcer and stomach cancer, and are contained in functional foods for protecting stomach and duodenum from the colonization of Helicobacter pylori.

Functional foods of the present invention are common foods such as suspension or beverage.

Functional foods of the present invention are prepared by adding antibiotic materials such as levulinic acid or its derivatives to common additives. Common additives are selected from a group comprising vitamin C, powdery vitamin E, iron lactate, zinc oxide, nicotinic acid amide, vitamin A, vitamin B₁, vitamin B₂ and their mixture. Contents of the common additives in functional foods are determined by taste, cutaneous sensation and consumer proclivity. Preferably, the common additives contain 0.001˜10% of the total weight of functional foods.

The present inventions will be explained in more detail with reference to the following examples. However, the following examples are provided only to illustrate the present invention, and the present invention is not limited to them.

### PREPARATION EXAMPLE 1

### Preparation of Tablet by Direct Compression

Levulinic acid (5.0 mg) was mixed with 14.1 mg of lactose, 0.8 mg of crosspovidon USNF and 0.1 mg of magnesium stearate. The resultant mixture was compressed, to give tablet.

The above tablet was comprising;

| | |
|---|---|
| levulinic acid | 5.0 mg |
| lactose | 14.1 mg |
| crosspovidon USNF | 0.8 mg |
| magnesium stearate | 0.1 mg |

### PREPARATION EXAMPLE 2

### Preparation of Tablet by Wet Granulation

Levulinic acid (5.0 mg) was mixed 16.0 mg of lactose and 4.0 mg of starch. Some of solution prepared by dissolving 0.3 mg of polysorbate 80 in distilled water was added therein. The resultant mixture was granulized, dried and sieved, to form granule. The granule was mixed with 2.7 mg of colloidal silicon dioxide and 2.0 mg of magnesium stearate. The above mixture was compressed to give tablet.

The above tablet was comprising;

| | |
|---|---|
| levulinic acid | 5.0 mg |
| lactose | 16.0 mg |
| starch | 4.0 mg |
| polysorbate 80 | 0.3 mg |
| colloidal silicon dioxide | 2.7 mg |
| magnesium stearate | 2.0 mg |

### PREPARATION EXAMPLE 3

### Preparation of Capsule

Levulinic acid (5.0 mg) was mixed with 14.8 mg of lactose, 10.0 mg of polyvinyl pyrrolidone and 0.2 mg of magnesium stearate. The resultant mixture was filled in gelatin capsule.

The above capsule was comprising;

| | |
|---|---|
| levulinic acid | 5.0 mg |
| lactose | 14.8 mg |
| polyvinyl pyrrolidone | 10.0 mg |
| magnesium stearate | 0.2 mg |

### PREPARATION EXAMPLE 4

### Preparation of Injectable Solution

Levulinic acid (100 mg) was mixed with 180 mg of mannitol, 26 mg of Na₂HPO₄.12H₂O and 2974 mg of distilled water. The mixture was prepared to injectable solution. The injectable solution was sterilized at 20° C. for 30 min.

The above injectable solution was comprising;

| | |
|---|---|
| levulinic acid | 100 mg |
| mannitol | 180 mg |
| Na₂HPO₄.12H₂O | 26 mg |
| distilled water | 2974 mg |

### PREPARATION EXAMPLE 5

### Preparation of Cream

Stearic acid, cetostearyl alcohol, caprylic capric triglyceride, mineral oil and butylene glycol were heated at 75° C., to give organic phase. Levulinic acid, distilled water, glycerin, tween 60, tween 80 and potassium hydroxide were mixed, to form aqueous phase. The organic phase was added to the aqueous phase. The mixture was stirred at 1200˜1500 rpm for 10˜20 min, cooled and disposed at the room temperature. Total weight of cream was 100 g.

The above cream was comprising;

| | |
|---|---|
| levulinic acid | 100.0 mg |
| stearic acid | 3.0 mg |
| cetostearyl alcohol | 2.0 mg |
| caprylic capric triglyceride | 5.0 mg |
| mineral oil | 8.0 mg |
| butylene glycol | 3.0 mg |
| glycerin | 6.0 mg |
| Tween 60 | 2.5 mg |
| Tween 80 | 1.0 mg |
| potassium hydroxide | 0.5 mg |
| distilled water | conditional-weight |

### <PREPARATION EXAMPLE 6

### Preparation of Beverage

Levulinic acid, vitamin C, powdery vitamin E, iron lactate, zinc oxide, nicotinic acid amide, vitamin A, vitamin B₁, vitamin B₂ was mixed, to give beverage.

The above beverage was comprising;

| | |
|---|---|
| levulinic acid | 0.1 g |
| vitamin C | 15 g |
| powdery vitamin E | 7.5 g |
| iron lactate | 19.75 g |
| zinc oxide | 3.5 g |
| nicotinic acid amide | 3.5 g |
| vitamin A | 0.2 g |
| vitamin B₁ | 0.25 g |
| vitamin B₂ | 0.3 g |
| distilled water | conditional-weight |

### EXPERIMENTAL EXAMPLE 1

### Test for Antibiotic Activity of Levulinic Acid

(Step 1) Culture of Testing Bacteria

Staphylococcus aureus KCTC 1621, Pseudomonas aeroginosa KCTC 1636, methicillin-resistant Staphylococcus aureus, Vancomycin resistant Enterococcus and R-Pseudomonas aeroginosa were cultured in tryptic soy broth at 37° C. with shaking.

Lactococcus garvieae YT-3, Edwardsiella tarda GY-01, Vibrio anguillarum YT-85805, Enterococcus faecium KCTC 3095 and tooth-decaying bacteria of Streptococcus spp. were cultured in Brain heart infusion media at 37° C. with shaking. Anaerobic tooth-decaying bacteria, Streptococcus spp., were cultured with standing in gas generating kit. Anaerobic Propionibacterium acnes KCTC 3326, was cultured in GAM media with standing in gas generating kit.

Also, Bacillus cereus KCTC 1014, Bacillus subtillis IFO 3007, Escherichia coli O 157 and Escherichia coli 1039 were cultured in complex media (0.8% of nutrient broth, 1% of yeast extract, 2% of NaCl and 0.02% of dextrose) at 37° C. with shaking.

Saccharomyces cerevisiae breneri hefe rasse VIII and Hansenula anomala IFO 0149 were cultured in complex medium (0.3% of yeast extract, 0.3% of nutrient broth, 0.5% of peptone and 0.1% of glucose) at 25° C. with shaking.

Candida albicans KCTC 7753 was shaking-cultured in complex medium made from 0.3% of yeast extract, 0.3% of malt extract, 0.5% of peptone and 1% dextrose at 25° C.

Helicobacter pylori was standing-cultured in complex medium (5.2% of brain heart extract, 0.1% of yeast extract, 5% of blood and 5% of horse serum) in anaerobic condition by gas generating kit at 37° C.

Wherein, all the bacteria were cultured for 12˜18 hours.

(step 2) Determination of MIC (Minimal Inhibitory Concentration) of Levulinic Acid

MIC of levulinic acid was measured on 96 well plates. Media and levulinic acid (200 mg/ml) mixed and diluted 1:2 fold serially. Dilutants were added on 96 well plate. Equal amount of bacterial suspension (10⁶ CFU/ml) was added in each well. Wherein the inoculated bacteria were cultured at 30° C. for 12˜18 hours. The proliferations of bacteria on testing medium were measured to determine MIC against the test bacteria.

Also, in case of anaerobic bacteria, according to the same method as above, cap tubes were used in place of 96 well plates. The results were represented in Table 1.

TABLE 1
MIC of levulinic acid against drug-resistant bacteria

| Bacteria | MIC (mg/ml) | Property |
|---|---|---|
| Staphylococcus aureus | 3.5 | Gram positive, Antibiotic |
| KCTC 1621 | | sensitive |
| methicillin-resistant | 4 | Gram positive, Separated |
| Staphylococcus aureus | | from Korean patient, |
| | | Drug-resistant bacteria |
| Pseudomonas aeruginosa | 1.5 | Gram negative, Antibiotic |
| KCTC 1636 | | sensitive |
| Resistant Pseudomonas | 2.5 | Gram negative, Separated |
| aeruginosa | | from Korean patient, |
| | | Drug-resistant bacteria |
| Enterococcus faecium | 4.5 | Gram negative |
| KCTC 3095 | | |
| vancomycin-resistant | 3 | Gram positive, Separated |
| Enterococcus | | from Korean patient, |

As shown in Table 1, levulinic acid was observed to have antibiotic activity and its MIC against drug-resistant bacteria were 1.5˜4.5 mg/ml. Particularly, antibiotic activity against Pseudomonas aeroginosa KCTC 1636 and R-Pseudomonas aeroginosa was excellent. Therefore levulinic acid was used as effective component of antibiotic against drug-resistant bacteria.

TABLE 2
MIC of levulinic acid against pathogen
infecting finny tribe and poultry

| Bacteria | MIC (mg/ml) | Property |
|---|---|---|
| Lactococcus garvieae | 3.5 | Gram positive, Separated |
| YT-3 | | from NFRDA, pathogen of |
| | | Streptococcus |
| Edwardsiella tarda | 3.5 | Gram negative, Separated |
| GY-01 | | from NFRDA, pathogen of |
| | | Edwards |
| Vibrio anguillarum | 3 | Gram negative, Separated |
| YT-85805 | | from NFRDA, pathogen of |
| | | Vibrio |
| Salmonella typhimurium | 0.37 | Gram negative, pathogen of |
| KCTC 1925 | | poultry |

As shown in Table 2, MIC against pathogen infecting finny tribe and poultry were 0.37˜3.5 mg/ml. Particularly, MIC towards Salmonella typhimurium KCTC 1925 was 0.37 mg/ml. Therefore, levulinic acid was used as effective component of antibiotic against pathogenic organism infecting finny tribe and poultry.

TABLE 3
MIC of levulinic acid against tooth-
decaying bacteria

| | MIC | |
|---|---|---|
| Bacteria | (mg/ml) | Property |
| Streptococcus mutans KCTC | 4 | Gram positive, |
| 3065 | | Anaerobic |
| Streptococcus mutans HS-6 | 4 | |
| Streptococcus mutans BHT | 4 | |
| Streptococcus mutans GS-5 | 5 | |
| Streptococcus mutans | 4 | |
| Ingbritt | | |
| Streptococcus mutans OMZ- | 5 | |
| 176 | | |
| Streptococcus mutans LM-7 | 4 | |
| Streptococcus mutans OMZ- | 4 | |
| 175 | | |
| Streptococcus sobrinus | 4 | |
| coykendall 6715-1 ATCC | | |
| 27352 | | |
| Streptococcus doweni | 4 | |
| Mfe28 | | |
| Streptococcus salivarius | 4 | |
| ATCC 13419 | | |

As shown in Table 3, MIC against tooth-decaying bacteria was 4˜5 mg/ml. Therefore, levulinic acid was used as effective component of antibiotic against tooth-decaying bacteria.

TABLE 4
MIC against general bacteria and yeast

| | MIC | |
|---|---|---|
| Bacteria | (mg/ml) | Property |
| Escherichia coli O157 | 2 | Gram negative, |
| | | pathogenic E. coli |
| Escherichia coli KCTC | 2 | Gram negative |
| 1039 | | pathogenic E. coli |
| Bacillus cereus KCTC | 1.5 | Gram positive |
| 1014 | | |
| Bacillus subtilis IF0 | 1.5 | Gram negative |
| 3007 | | |
| Saccharomyces | 2.5 | Yeast |
| cerevisiae breneri heferasse | | |
| VII | | |
| Hansenula anomala IFO | 3.5 | Yeast |
| 0149 | | |
| Candida albicans KCTC | 6 | Yeast |
| 7753 | | |

As shown in table 4, MIC against general bacteria and yeast were 1.5˜6 mg/ml. It was observed that levulinic acid had excellent antibiotic activity against various bacteria and yeast.

TABLE 5
MIC against bacteria causing pimple and
stomach ulcer

| Bacteria | MIC | Property |
|---|---|---|
| Propionibacterium | 6 mg/ml | Gram positive, Anaerobic |
| acnes KCTC 3326 | | Pimple causing bacteria |
| Helicobacter | 0.78 μg/ml | Gram negative bacteria causing |
| pylori | | stomach ulcer, duodenal ulcer |
| | | and stomach cancer |

As shown in Table 5, MIC against pimple causing bacteria was 6 mg/ml. MIC against bacteria causing stomach ulcer, duodenal ulcer and stomach cancer were 0.78 μg/ml.

### EXPERIMENTAL EXAMPLE 2

### Test for Cytotoxicity of Levulinic Acid

Cytotoxicity test of levulinic acid was accomplished by using mouse fibroblast NIH3T3 cells as testing cells. The cells were cultured in medium added 16.3 mg/ml, 12.2 mg/ml, 9.8 mg/ml and 4.9 mg/ml of levulinic acid for 52 hours, respectively.

The cells cultured in medium containing 16.3 mg/ml and 12.2 mg/ml of levulinic acid were died, or their growth were inhibited. The cells cultured in medium added 9.8 mg/ml of levulinic acid were not died, but their growth were inhibited. Growth of the cells cultured in medium containing 4.9 mg/ml of levulinic acid has no difference from one of control cells cultured in medium added no levulinic acid. Therefore it was observed that formulation tested levulinic acid (5.0%) is safe enough for other application without damaging normal host cells.

### EXPERIMENTAL EXAMPLE 3

### Acute Toxicity in Rats Via Oral Administration

The following experiment was performed to see if levulinic acid has acute toxicity.

Six-week old specific-pathogen free (SPF) SD rats were used for the acute toxicity examination. Formulations prepared in preparation example 1˜3 were suspended in methyl cellulose solution and orally administered once to two rats per group at the dosage of 10 mg/kg/15 ml.

After administration, death, clinical symptoms and weight change in the tested rats were monitored. In addition, hematological and biochemical tests in blood were performed, and any abnormal signs in the gastrointestinal organs of chest and abdomen were visually (with the naked eye) checked during autopsy.

The results showed that the tested formulations did not cause any specific clinical symptoms, weight changes, or death in rats. No change was observed in hematological tests, biochemical tests and autopsy. Therefore, the formulations used in this experiment are evaluated to be safe, since they do not cause any toxic change in rats up to the level of 2 g/kg and their minimum lethal dose (LD₅₀) is much higher than 100 mg/kg.

### EXPERIMENTAL EXAMPLE 4

### Topical Toxicity in Rats

The following experiment was performed to determine if creams prepared in preparation example 5 have topical toxicity.

Six-week old hairless mouse (SKH-1) were used in the tests of topical toxicity, according to regulation of KFDA. The creams were applied to two rats per group at the concentration of 1 g/cm, once a day for 20 days.

The results showed that the tested creams did not cause topical toxicity in rats. Therefore, the creams are evaluated to be non-toxic at the common concentration.

As stated above, in accordance with the present invention, levulinic acid and its derivatives have considerable antibiotic activity against Gram positive and negative bacteria, yeast, drug-resistant bacteria and common-antibiotic-resistant bacteria such as methicillin-resistant Staphylococcus aureus, R-Pseudomonas aeruginosa and vancomycine-resistant Enterococcus. Therefore levulinic acid and its derivatives are used as antibiotics for human and animal, and used as effective ingredients of functional cosmetics and functional foods.

What is claimed is:

1. A method of treating bacterial infections comprising:
administering an effective amount of an antibiotic composition of the formula 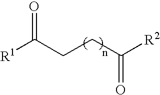
wherein
R¹ is C₁˜C₄ straight or branched alkyl group, or C₃˜C₇ cycloalkyl group;
R² is selected from the group consisting of OH, C₁˜C₄ straight or branched alkoxy group, —NH₂, and amine substituted with C₁˜C₄ straight or branched alkyl group or C₅˜C₆ methylene group; and
n is an integer 1 to 3
to a patient in need thereof.

2. The method of claim 1, wherein the antibiotic composition is administered in amounts ranging from 1 mg to 10 g per day.

3. The method of claim 1, wherein the antibiotic composition is administered in amounts ranging from 1 mg to 5 g per day.

4. The method of claim 1, wherein R¹ is CH₃, R² is OH and n is 1.
